(19) United States
(12) Patent Application Publication
(10) Pub. No.: US 2009/0177271 A1
(43) Pub. Date: Jul. 9, 2009

(54) Prosthesis for Ascending Aortic Segment and Method for Surgical Treatment of an Aortic Valve Leak

(76) Inventor: Jean-Noel Fabiani, Paris (FR)

Correspondence Address: OLIFF & BERRIDGE, PLC, P.O. BOX 320850, ALEXANDRIA, VA, 22320-4850, US

(21) Appl. No.: 11/992,648

(22) PCT Filed: Sep. 29, 2006
(86) PCT No.: PCT/FR2006/002210

§ 371 Date: Jun. 10, 2008

(30) Foreign Application Priority Data
Sep. 29, 2005 (FR) 0509954

(51) Int. Cl. A61F 2/06; A61F 2/24

(52) U.S. Cl. 623/1.26; 623/2.38; 623/2.4

(57) ABSTRACT

The invention concerns a method for preauthentication of a first entity (10) by a second entity (1) communicating with each other through a wireless link, including the following steps: the second entity (1) emits (23′) a challenge value (c); if the first entity (10) receives (23) a challenge value (c′) it applies to the received challenge value a predefined transformation (g) known to the second entity, to obtain a first transformed value (r), then emits (24) the first transformed value obtained; and if the second entity receives (24′) a transformed value (r′) it compares (25′) the received transformed value to a second transformed value (r″) obtained by applying the predefined transformation (g) to the emitted challenge value, and estimates the preauthentication to be successful if the result of the comparison between the second transformed value obtained and the received transformed value is less than a predefined threshold (m).

The present invention concerns a prosthesis for an ascending aortic segment and a method for surgical treatment of an aortic valve leak.

The aorta is the largest artery in the human body, originating from the heart and travelling all along the anterior aspect of the spine. From there, numerous arteries carry blood throughout the body.

The opening in the aorta nearest the heart is closed by a valve consisting of three aortic valves (posterior valve, left anterior valve and right anterior valve) which are membranous or sigmoid (i.e. pigeon-nest) folds shaped like half-baskets with one convex side. Each of them has an Arantius' nodule at its upper tip. The joining of the three Arantius' nodules ensures total closure of the opening.

The leaflets of the aortic valve separate under systolic pressure and come together under diastolic pressure (when the blood flow is reversed) to close the corresponding opening and avoid reflux.

In doing so, they provide a seal for the ventricles in diastole (relaxation), thereby avoiding the flow of blood back from the ascending aorta into the left ventricle. However, in some patients the valve dilates and the aortic leaflets, which are then separated from each other, no longer close the opening correctly.

Incomplete closure of the aortic valve leads to blood flow back from the aorta into the left ventricle under diastolic pressure, leading to the dilation of the ventricle; this is known as an aortic valve leak and its immediate consequence is haemodynamic insufficiency caused by the excess blood in the ventricle.

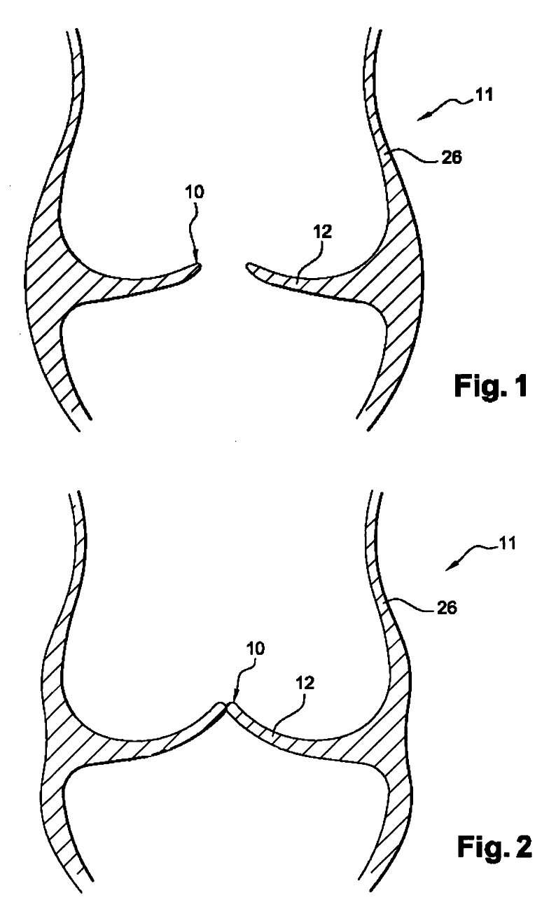

This situation is illustrated in FIGS. 1 and 2. The Arantius' nodules 10, which should completely close off the aorta 11, can be clearly seen. The nodules control aortic leaflets 12, which are normally shaped like a half-basket.

In the configuration shown in FIG. 1, the leaflets of the aortic valve 12 have lost their convexity and can therefore no longer close correctly

In the configuration shown in FIG. 2, the aortic leaflets 12 close correctly.

To treat haemodynamic insufficiency, a surgeon may decide to replace the aortic valve or repair the existing, malfunctioning valve.

However, if at all possible, surgeons prefer to preserve existing aortic valves (this is known as reparative surgery or valvuloplasty) when their condition allows it.

The existing technique illustrated by document FR-A-2688692 is already used to repair native aortic valves.

To carry out such a repair, a wire frame reproducing the trifoliate shape of the aortic valve opening is used. The wire frame is fitted with a string that is used to connect the cage to the valve opening at the base of the aortic valves by means of sutures. The operation enables the aortic valves 12 to be tightened and to regain their half-basket shape.

However, after a valve leak, the ascending segment of the aorta often no longer maintains arterial pressure, creating an aneurysm, a dilation of a segment of the aorta resulting from structural changes in its wall.

In this case, the patient has to undergo a second operation and an ascending valve segment prosthesis is inserted.

At present, when treating such cases, an ascending aortic segment prosthesis is assembled with a mechanical valve prosthesis or a bioprosthesis. However, a mechanical prosthesis implant, as shown in WO 95/03754, requires post-operative treatment with anti-coagulants, a treatment that may be restrictive for certain categories of patient (children or adolescents, young women wishing to have children etc.).

It is impossible to implant a combination of bioprosthesis and ascending aortic segment prosthesis because of incompatibilities in the conservation of these two types of prosthesis (synthetic Dacron fibres and biological matter) outside the human body. A combination implant therefore poses difficulties. To solve this problem, an alternative is currently being sought to the aortic segment prosthesis made of Dacron.

The primary aim of the invention is to solve the problem by providing an ascending aortic valve prosthesis that will allow for a valvuloplasty and prevent aortic aneurysm.

The invention concerns a prosthesis for an ascending aortic segment valve consisting of a tube having one end cut out to match the trifoliate shape of a valve opening. The tube is secured, at the cut out end, to a wire frame extending along a closed outline substantially shaped like a healthy native aortic valve ring. The wire frame is linked to a suture zone extending along the wire-shaped frame over the whole length of the closed outline.

The invention provides a new solution to the problem of treating valve leaks while preventing an aortic aneurysm. According to the invention, the prosthesis may be built entirely of synthetic material e.g. Dacron and can be preserved outside the human body. One major consequence for the patient is that the time required for surgical treatment may be shortened. Moreover, the implant of the prosthesis described in the invention causes little damage to the body because it does not include any traumatic surgery such as the dissection of the coronary arteries.

Also, the prosthesis described in the invention allows existing valves to be preserved and this avoids frequent changes of prosthesis throughout the patient's life. More importantly, it avoids a wide range of post-operative treatments such as the prescription of anti-coagulants which are necessary after the implanting of artificial valves. The risk of complications due to poor compliance with the post-operative treatment is therefore reduced. In operations on young children, for whom the prosthesis described in the invention should be replaced after a few years to allow for the growth of the aorta, the replacement can be undertaken later than with a prosthetic valve because the wire frame does not obstruct the aorta when the valve leaflets open, contrary to the case with artificial prostheses.

Another advantage is that the ascending aortic segment prosthesis is similar to a Valsalva prosthesis, with a widened base similar in form to the Valsalva's sinuses.

Another characteristic is the optional fixation of the wire frame to the tip of the prosthetic tube by sutures or welding.

The invention also aims to provide a method of surgical treatment for an aortic valve leak caused by a diseased aortic ring. The invention consists in implanting a prosthesis as described above, consisting of a wire frame following a closed contour and with a similar shape to a native aortic valve ring, and to suture onto a diseased native aortic ring, the suture zone being connected to the prosthesis frame.

Other characteristics of the prosthesis described in the invention are as follows
the wire frame is sheathed in polyester fibres;
the polyester fibres are of the same type as those marketed under the Dacron brand, which is the property of Dupont de Nemours;
the wire frame is flexible, to make it easier to insert into the aorta;
the wire frame is made of a stainless steel alloy of the type marketed under the Elgiloy brand by the Elgiloy Corporation;
the suture zone is a Dacron string;
the wire frame is held in place in the suture zone by a Dacron envelope

The invention is easier to understand after reading the following description, given solely as an example and with reference to the drawings in which:

FIG. 1 is a cross-section of a diseased aortic valve;

FIG. 2 is a cross-section of a healthy aortic valve;

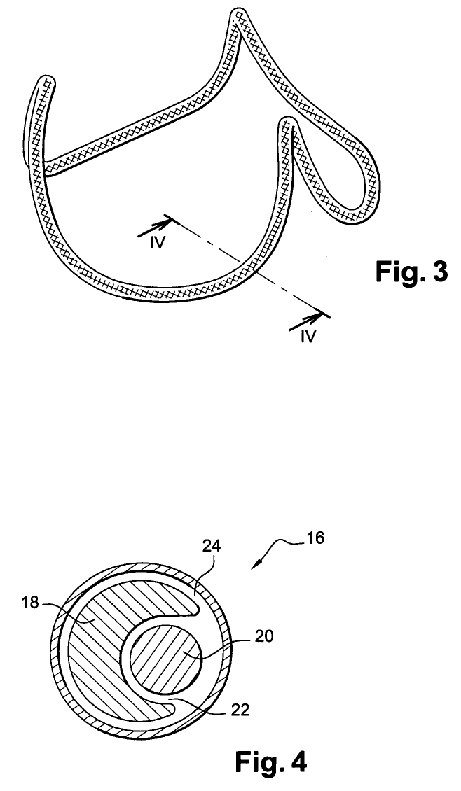

FIG. 3 shows, in perspective, a sheathed wire frame forming a ring prosthesis for an aortic valvuloplasty according to one method of use for the invention;

FIG. 4 is a cross-section along IV-IV of the sheathed wire frame as shown in FIG. 3;

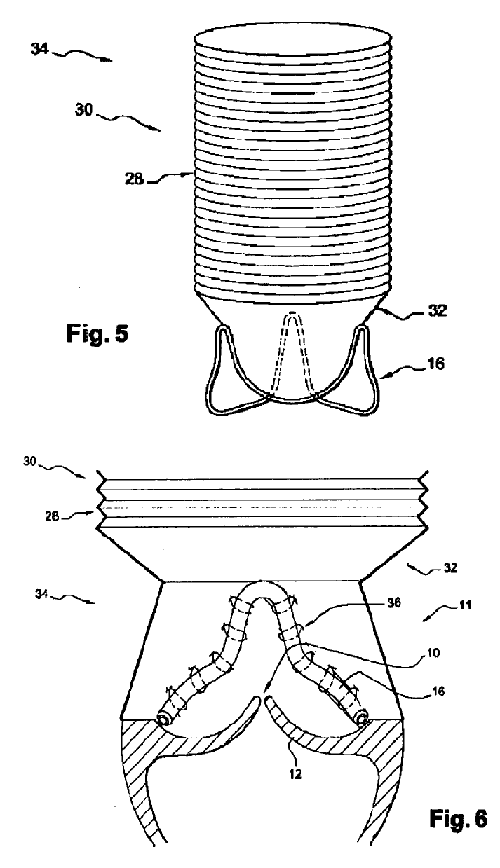

FIG. 5 shows a prosthesis implanted using one method for the invention comprising the wire frame from FIG. 3;

FIG. 6 shows the prosthesis from FIG. 5 in position on a diseased native aortic ring.

The description below refers to FIGS. 3 to 6, a wire frame 18. On FIGS. 3 to 6, the components are analogous to the ones shown in FIGS. 1 and 2 and bear identical references

A wire frame 18 forms a closed outline substantially shaped like a native ring in a healthy aortic valve.

The wire frame 18 is made of a stainless steel alloy such as the one marketed under the Elgiloy brand by the Elgiloy Corporation.

As shown in FIG. 4, a suture zone 20 consisting of a Dacron string runs along the wire frame 18 which includes a longitudinal groove 22 for this purpose in its circular section. The suture zone 20 permits the surgical stitching to connect the wire frame 16 to diseased native aortic valves.

The suture zone 20 and the wire frame 18 are connected to each other all round the closed outline by a Dacron envelope 24.

The flexible wire frame 18 sheathed in polyester fibres forms a wire frame 16.

As shown in FIG. 5, the wire frame 16 is linked to a tube 28, forming an ascending aortic segment prosthesis 34.

The tube 28 comprises an upper Dacron section 30 that is substantially tubular and a lower conical section 32 which is adapted to the diameter of the upper section 30 and the diameter of the wire frame 16.

One of the lower extremities (cf. the sketch) on the lower section 32 is shaped in accordance with the same closed contour as the wire frame 16 and is connected to the said wire frame 16 by sutures or welding.

The prosthesis 34 including the sheathed wire frame 16 and the tube 28 ensures a uniform reduction of the dilated aortic valve and the replacement of the diseased aortic segment.

FIG. 6 shows a method of surgical treatment for an aortic valve leak using the prosthesis 34.

After removing the ascending aortic segment, the prosthesis 34 is implanted by applying the wire frame 16 against the diseased native aortic ring 11. The wire frame 16 is then sutured to the said ring round the entire closed outline using knots 36.

Thanks to the frame, the aortic valves 12 regain their convexity. Moreover, the tube 28 replaces the ascending aortic segment that has been removed, preventing any risk of an aortic aneurysm.

The sheathed wire frame 16 is connected to the inside of the valve, which means that the aorta 11 does not have to be sectioned.

It is well understood that the surgical methods described above are by no means restrictive and can be modified in any way deemed preferable without moving out of the framework of the invention.

1. Prosthesis for an ascending aortic segment, consisting of a tube having one end cut out to match the trifoliate shape of a valve opening, the tube being secured, at this end, to a wire frame extending along a closed outline substantially shaped like the ring of a healthy native aortic valve, the wire frame being linked to a suture zone extending along the entire length of the closed outline of the wire frame.

2. Prosthesis in accordance with claim 1 in which the wire frame is sheathed with polyester fibres.

3. Prosthesis in accordance with claim 1, in which the polyester fibres are of the type marketed under the Dacron brand, being the property of the Dupont de Nemours company.

4. Prosthesis in accordance with claim 1, in which the wire frame is flexible to make it easier to insert in the aorta.

5. Prosthesis in accordance with claim 1, in which the wire frame is made of a stainless steel alloy such as the one marketed under the Elgiloy brand by the Elgiloy Corporation.

6. Prosthesis in accordance with claim 1, in which the suture zone consists of a Dacron string.

7. Prosthesis in accordance with claim 1, in which the wire frame is firmly attached to the suture zone by a Dacron envelope.

8. Prosthesis in accordance with claim 1, in which the tube is similar to a Valsalva prosthesis.

9. Prosthesis in accordance with claim 1, in which the wire frame is firmly attached to the tube by sutures or welding.

10. Surgical procedure to treat an aortic valve leak caused by a diseased aortic ring, consisting in a step of implanting a prosthesis in accordance with claim 1, including, in particular, a wire frame extending round a closed outline similar in shape to the ring of a native aortic valve, and in a step of suturing the suture zone attached to the wire frame of the prosthesis sutured onto the diseased native aortic ring.
